(19) United States
(12) Patent Application Publication
(10) Pub. No.: US 2026/0294512 A1
(43) Pub. Date: Oct. 1, 2026

(54) TECHNOLOGIES FOR OPERATING AN ENERGY-BASED SURGICAL INSTRUMENT BASED ON A TISSUE FINGERPRINT

(71) Applicant: Cilag GmbH International, Zug (CH)

(72) Inventors: Frederick E. Shelton, IV, Hillsboro, OH (US); Jason L. Harris, Lebanon, OH (US)

(21) Appl. No.: 19/344,320

(22) Filed: Sep. 29, 2025

Related U.S. Application Data
(60) Provisional application No. 63/780,377, filed on Mar. 30, 2025.

(51) Int. Cl. A61B 18/14; A61B 90/00; A61B 18/00

(52) U.S. Cl. CPC A61B 18/1445; A61B 90/06; A61B 2018/00642; A61B 2018/00702; A61B 2018/00875; A61B 2018/00982; A61B 2018/00994; A61B 2018/1452; A61B 2090/064

(57) ABSTRACT

Technologies for operating an energy-based surgical instrument based on a tissue fingerprint includes generating an impedance fingerprint of patient tissue grasped within a jaw assembly of an end effector of the energy-based surgical instrument. The impedance fingerprint is indicative of an impedance response of the patient tissue to different amounts of clamp pressure on the patient tissue by the jaw assembly. Additionally, an operational characteristic of the energy-based surgical instrument may be adjusted as a function of the impedance fingerprint.

This application claims the benefit of and priority to U.S. Patent Application No. 63/780,377, entitled “TECHNOLOGIES FOR OPERATING AN ENERGY-BASED SURGICAL INSTRUMENT BASED ON A TISSUE FINGERPRINT,” which was filed on Mar. 30, 2025, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient’s tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient’s tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, a method for operating an energy-based surgical instrument includes generating, by a controller of the energy-based surgical instrument, a tissue impedance fingerprint of patient tissue grasped within a jaw assembly of an end effector of the energy-based surgical instrument, wherein the tissue impedance fingerprint is indicative of an impedance response of the patient tissue to different amounts of clamp pressure on the patient tissue by the jaw assembly; adjusting, by the controller, an operational characteristic of the energy-based surgical instrument as a function of the tissue impedance fingerprint; and activating, by the controller, an operation of the energy-based surgical instrument using the adjusted operational characteristic.

In some embodiments, generating the tissue impedance fingerprint may include determining, by the controller, a clamp pressure exerted on the patient tissue by the jaw assembly and determining, by the controller, an impedance value of the patient tissue at the clamp pressure exerted on the patient tissue by the jaw assembly. In such embodiments, determining the clamp pressure may include applying, by the jaw assembly, an increasing clamp pressure on the patient tissue over a time period and determining, by the controller, a clamp pressure exerted on the patient tissue by the jaw assembly at multiple points in time within the time period. In such embodiments, determining the impedance value of the patient tissue may include determining, by the controller, an impedance value of the patient tissue at each point in time correlating to a determined clamp pressure.

Additionally, in some embodiments, determining the clamp pressure may include applying, by the jaw assembly, a sequence of random clamp pressures on the patient tissue of the patient tissue and determining, by the controller, a clamp pressure exerted on the patient tissue by the jaw assembly for each random clamp pressure of the sequence of random clamp pressures. In such embodiments, determining the impedance value of the patient tissue may include determining, by the controller, an impedance value of the patient tissue for each random clamp pressure of the sequence of random clamp pressures.

In some embodiments, determining the clamp pressure may include receiving, by the controller, sensor data from a clamp force sensor of the energy-based surgical instrument. The sensor data may be indicative of the clamp pressure. determining the clamp pressure may also include determining, by the controller, the clamp pressure exerted on the patient tissue by the jaw assembly based on the sensor data.

Additionally, in some embodiments, adjusting the operational characteristic of the energy-based surgical instrument may include adjusting a power level setting of the energy-based surgical instrument as a function of the tissue impedance fingerprint. Additionally or alternatively, in some embodiments, adjusting the operational characteristic of the energy-based surgical instrument may include adjusting an energy application modality of the energy-based surgical instrument as a function of the tissue impedance fingerprint. Further, in some embodiments, adjusting the operational characteristic of the energy-based surgical instrument may include adjusting a control point of an application of energy from the energy-based surgical instrument as a function of the tissue impedance fingerprint. Additionally or alternatively, in some embodiments, adjusting the operational characteristic of the energy-based surgical instrument may include adjusting a length of time for which the jaw assembly applies a clamp pressure to the patient tissue as a function of the tissue impedance fingerprint. Additionally or alternatively, in some embodiments, adjusting the operational characteristic of the energy-based surgical instrument may include adjusting a power level setting of an energy application of the energy-based surgical instrument as a function of the tissue impedance fingerprint while the energy application is being performed.

According to another aspect of the present disclosure, an energy-based surgical instrument may include an end effector, a clamp force sensor, and a controller. The end effector may have a jaw assembly configured to grasp patient tissue. The clamp force sensor may be configured to generate sensor data indicative of an amount of clamp pressure applied by the jaw assembly on the patient tissue when the patient tissue is grasped by the jaw assembly. The controller may be configured to generate a tissue impedance fingerprint of the patient tissue, wherein the tissue impedance fingerprint is indicative of an impedance response of the patient tissue to different amounts of clamp pressure on the patient tissue by the jaw assembly; adjust an operational characteristic of the energy-based surgical instrument as a function of the tissue impedance fingerprint; and activate an operation of the energy-based surgical instrument using the adjusted operational characteristic.

In some embodiments, to generate the tissue impedance fingerprint may include to determine a clamp pressure exerted on the patient tissue by the jaw assembly based on the sensor data and to determine an impedance value of the patient tissue at the clamp pressure exerted on the patient tissue by the jaw assembly. In such embodiments, to determine the clamp pressure may include to control the jaw assembly to apply, by the jaw assembly, an increasing clamp pressure on the patient tissue over a time period and to determine a clamp pressure exerted on the patient tissue by the jaw assembly at multiple points in time within the time period based on the sensor data. Additionally, in such embodiments, to determine the impedance value of the patient tissue may include to determine an impedance value of the patient tissue at each point in time correlating to a determined clamp pressure.

In some embodiments, to determine the clamp pressure may include to control the jaw assembly to apply, by the jaw assembly, a sequence of random clamp pressures on the patient tissue of the patient tissue and determine a clamp pressure exerted on the patient tissue by the jaw assembly for each random clamp pressure of the sequence of random clamp pressures based on the sensor data. In such embodiments, to determine the impedance value of the patient tissue may include to determine an impedance value of the patient tissue for each random clamp pressure of the sequence of random clamp pressures.

Additionally, in some embodiments, to adjust the operational characteristic of the energy-based surgical instrument may include to adjust a power level setting of the energy-based surgical instrument as a function of the tissue impedance fingerprint. Additionally or alternatively, in some embodiments, to adjust the operational characteristic of the energy-based surgical instrument may include to adjust an energy application modality of the energy-based surgical instrument as a function of the tissue impedance fingerprint.

In some embodiments, to adjust the operational characteristic of the energy-based surgical instrument may include to adjust a control point of an application of energy from the energy-based surgical instrument as a function of the tissue impedance fingerprint. Additionally or alternatively, to adjust the operational characteristic of the energy-based surgical instrument may include to adjust a length of time for which the jaw assembly applies a clamp pressure to the patient tissue as a function of the tissue impedance fingerprint. Furthermore, in some embodiments, to adjust the operational characteristic of the energy-based surgical instrument may include to adjust a power level setting of an energy application of the energy-based surgical instrument as a function of the tissue impedance fingerprint while the energy application is being performed.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a block diagram of another embodiment of a control circuit of the surgical instrument of FIG. 2;

FIG. 9 is a simplified flow diagram of a method for controlling an energy-based surgical instrument based on a tissue impedance fingerprint of a patient tissue;

FIG. 10 is a simplified flow diagram of a method for determining a tissue impedance fingerprint of a patient tissue; and

FIG. 11 is an illustrative graph of an embodiment of a tissue impedance fingerprint.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient’s natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

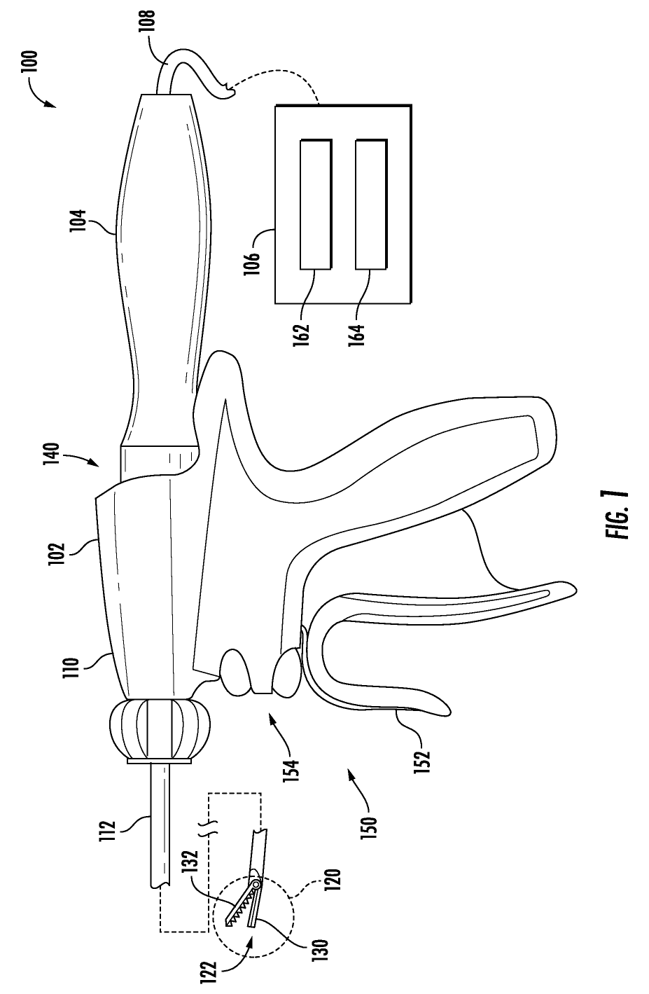

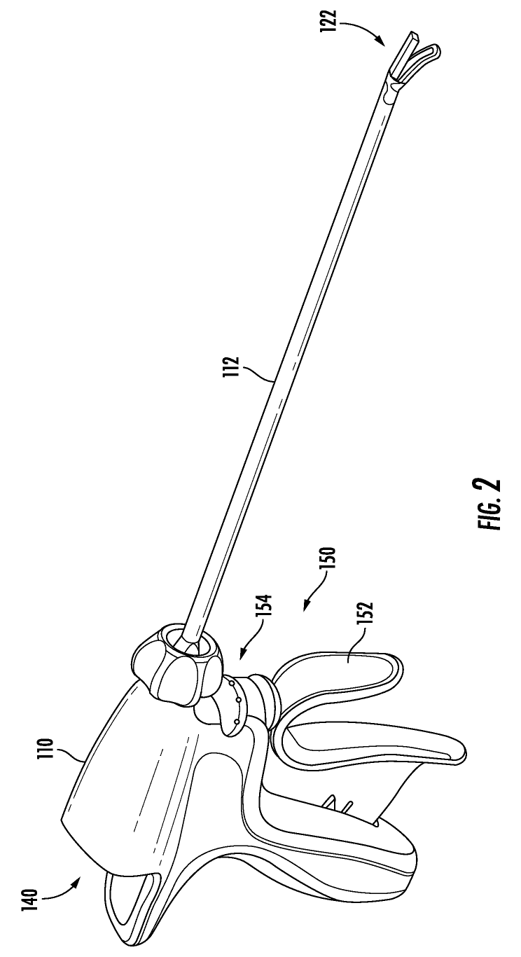

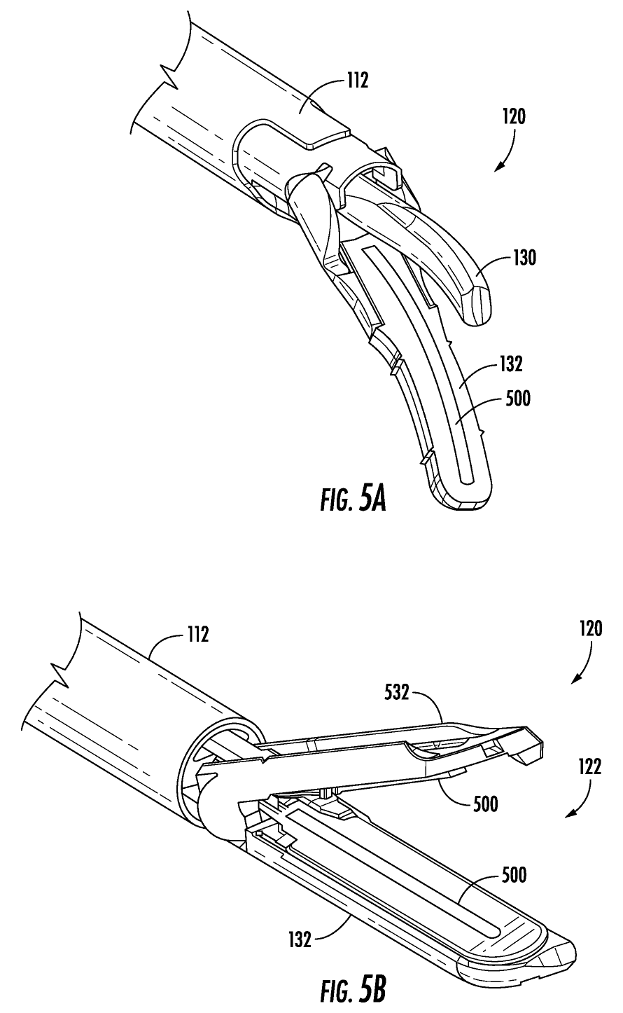

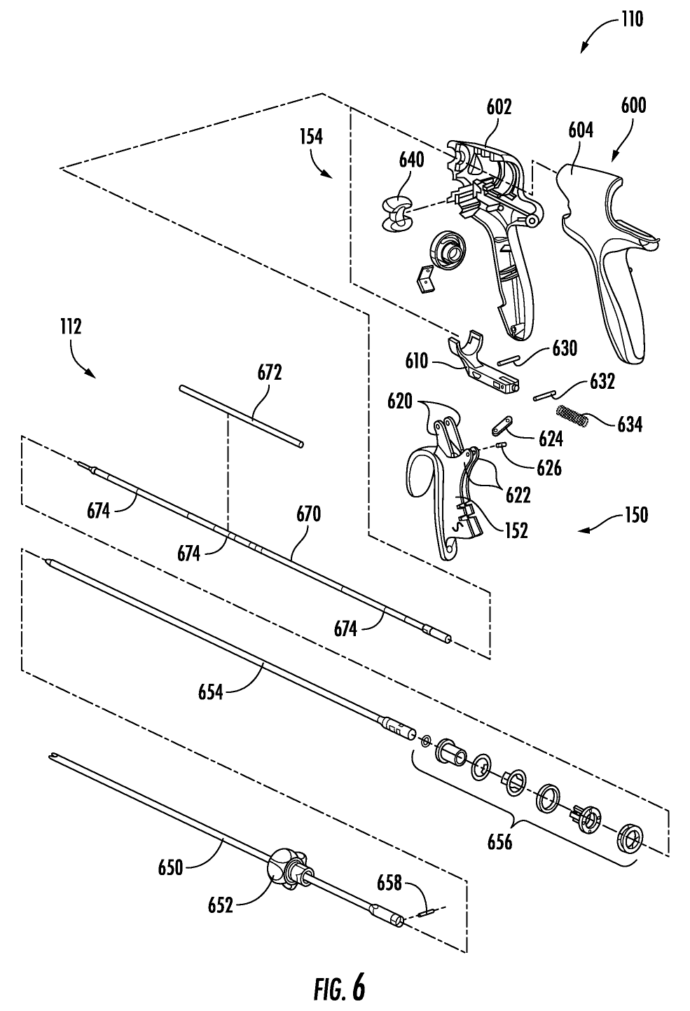

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

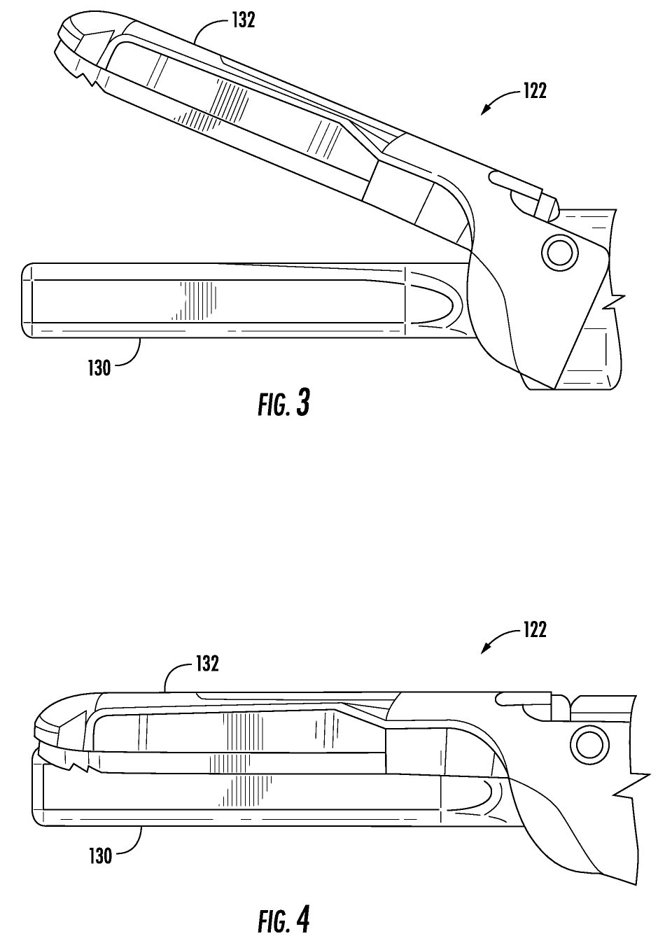

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both an ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient’s body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient’s tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient’s tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient’s tissue. For example, the electro-surgical generator module 164 may be configured to measure an impedance of the patient’s tissue using the electrodes 500 and a suitable RF energy level.

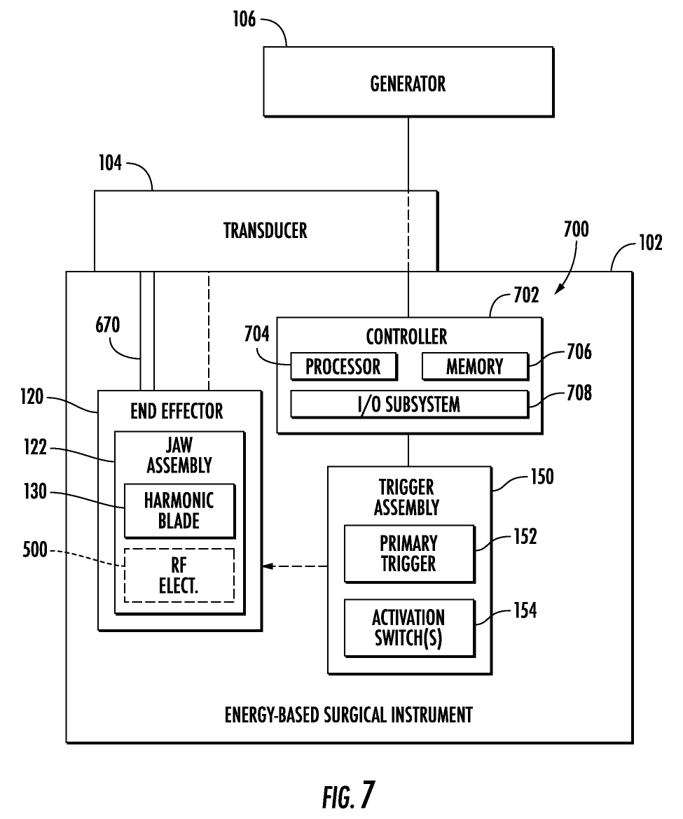

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

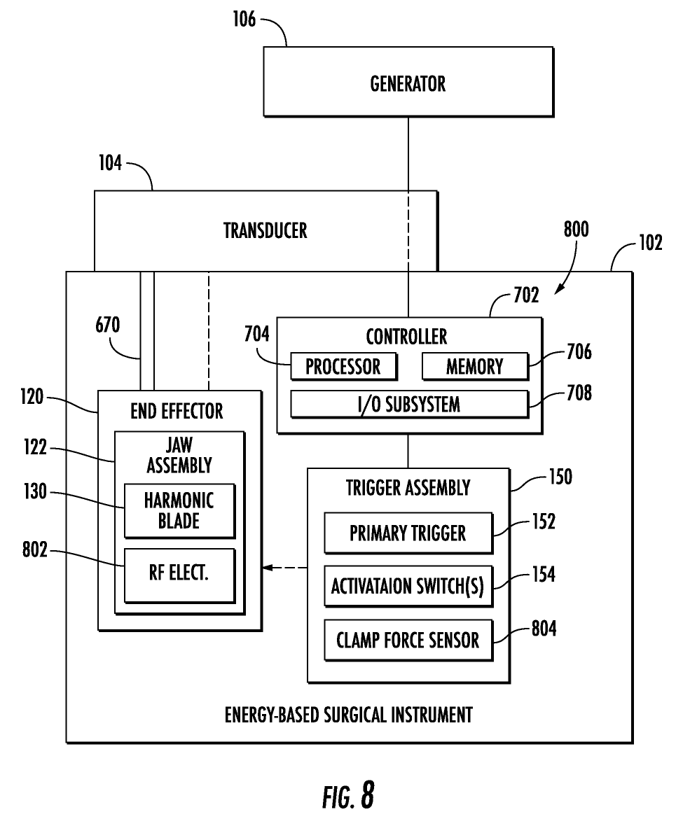

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

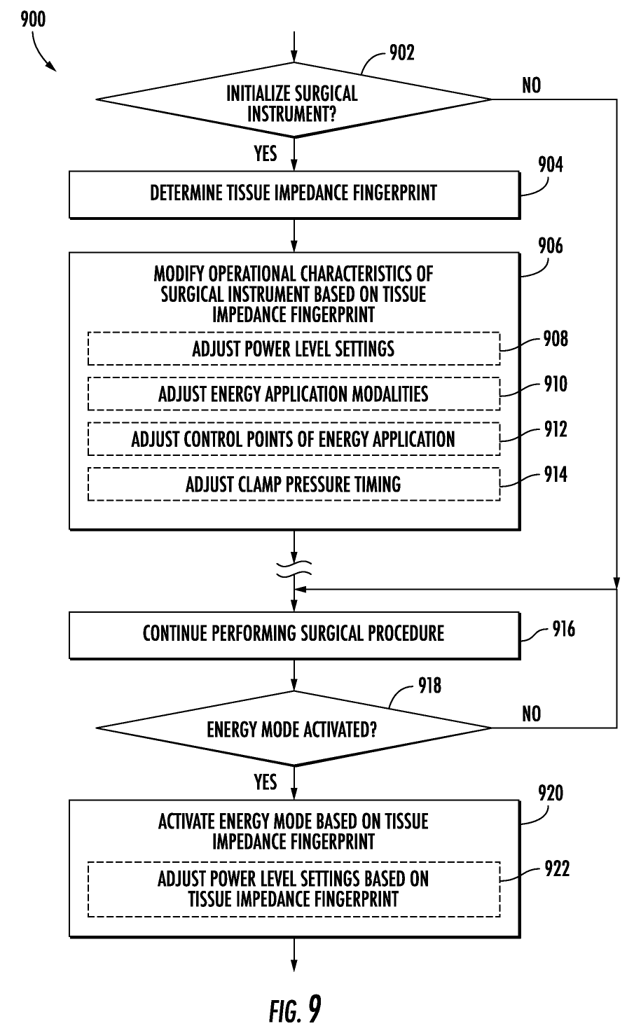

Referring now to FIG. 8, in another embodiment, the surgical instrument 102 is configured to determine a tissue impedance fingerprint of patient tissue grasped by the jaw assembly 122 and adjust one or more operational characteristics based on the determined tissue impedance fingerprint. Generally, total tissue impedance is a result of internal tissue aspects (e.g., conductivity, resistance, salinity, collagen content, elastic content, water content, fat percentage, ionic level of associated fluids, lattice of the tissue, etc.), the pressure applied to the tissue, and the power level of the energy applied. The tissue impedance fingerprint is indicative of an impedance response of the patient tissue to different amounts of clamp pressure applied to the patient tissue by the jaw assembly 122. For example, the tissue impedance fingerprint may be embodied as a correlated set of impedance and pressure values or as a graph of tissue impedance versus clamp pressure. The tissue impedance fingerprint provides a more defined response relationship of the tissue to the clamp pressures. The tissue impedance fingerprint may be used by the surgical instrument 102 to, for example, determine the type of tissue and/or tissue parameters such as calcification of the tissue, chronic remodeling of the tissue, extent of external forces (e.g., chemotherapy or radiation) on the tissue, ionic solution changes, and so forth. In the illustrative embodiment, the surgical instrument 102 may be configured to modify one or more operational characteristics as a function of the determined tissue impedance. For example, the surgical instrument 102 may be configured to adjust a power level setting, adjust an energy application modality, adjust a control point of an application of energy by the surgical instrument 102, adjust a length of time for which the jaw assembly 122 compresses the patient’s tissue, and/or other operational characteristics of the surgical instrument.

In such embodiments, the surgical instrument 102 may include a control circuit 800 as shown in FIG. 8. The control circuit 800 is substantially similar to the control circuit 700 described above and components of the control circuit 800 that correspond to similar components of control circuit 700 have been identified using the same reference number in FIG. 8 with the understanding that the description of such components provided above with regard to FIG. 7 applies to the corresponding components of the control circuit 800.

As shown in FIG. 8, the control circuit 800 includes a radio frequency (RF) electrode 802 and a clamp force sensor 804. The RF electrode 802 is included in the jaw assembly 122 of the end effector 120 and is substantially similar to the RF electrodes 500 described above. That is, the RF electrode 802 is operable to apply an amount of RF energy to patent tissue grasped by the jaw assembly 122. The RF electrode 802 may apply a therapeutic level of RF energy sufficient to, for example, create a hemostatic seal of the patient tissue. Additionally, the RF electrode 802 may apply a sub-therapeutic level of RF energy to perform various electrical measurements of the patient’s tissue, including an impedance measurement via the application of a suitable voltage and measurement of the resulting current, for example.

The clamp force sensor 804 may be embodied as any type of sensor capable of generating sensor data indicative of an amount of clamp pressure asserted on the patient’s tissue by the jaw assembly 122. For example, the clamp force sensor 804 may be embodied as a piezoelectric pressure sensor, a strain gauge, or other type of sensor capable of generating sensor data indicative of the clamp pressure applied by the jaw assembly 122 or from which such clamp pressure can be determined. As discussed in more detail below, the illustrative energy-based surgical instrument 102 is configured to apply various amounts of clamp pressure to the captured patient tissue via the jaw assembly 122 and measure the resulting clamp pressure using the clamp force sensor 804.

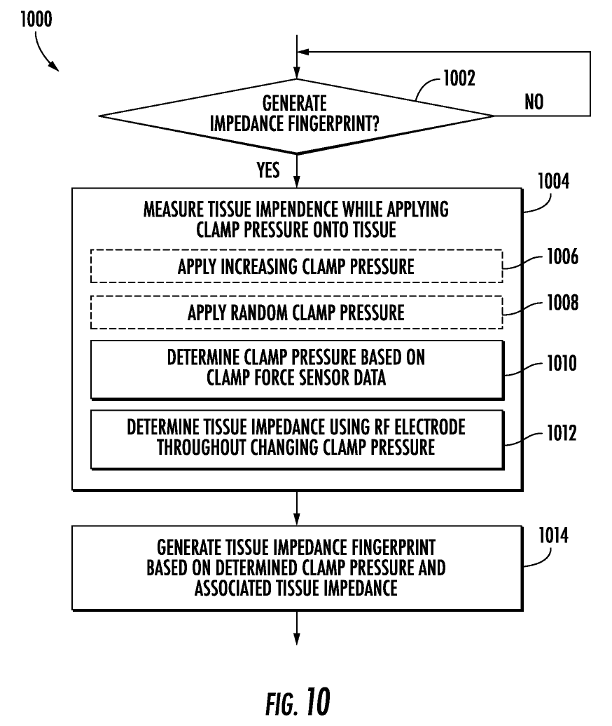

Referring now to FIG. 9, in use, the controller 702 of the energy-based surgical instrument 102 may execute a method 900 for controlling an energy-based surgical instrument based on a tissue impedance fingerprint of a patient tissue. The method 900 begins with block 902 in which the controller 702 determines whether to initialize the surgical instrument 102 based on the patient’s tissue. That is, as described below, one or more operational characteristics may be adjusted or pre-set based on a determined tissue impedance fingerprint of the patient tissue.

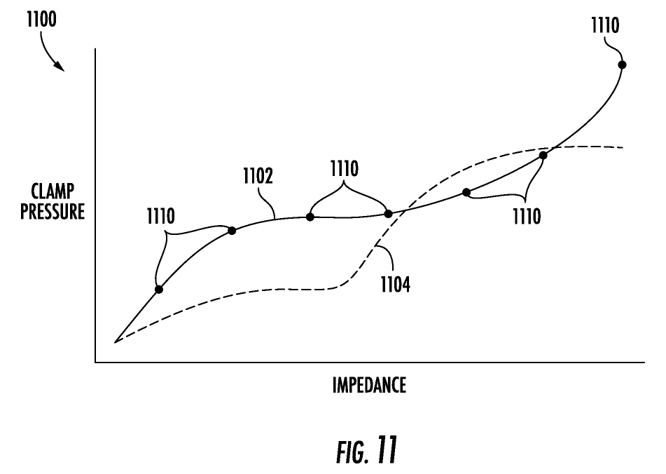

If the controller 702 determines to initialize the surgical instrument 102 based on the tissue impedance fingerprint of the patient tissue, the method 900 advances to block 904. In block 904, the controller 702 determines a tissue impedance fingerprint of the patient tissue presently grasped within the jaw assembly 122. To do so, the controller 702 may execute a method 1000 as shown in FIG. 10. The method 1000 begins with block 1002 in which the controller 702 determines whether to generate a tissue impedance fingerprint.

Again, if so, the method 1000 advances to block 1004 in which the controller 702 measures a tissue impedance of the patient’s tissue while applying varying or multiple clamp pressures (e.g., stepped pressure amounts) to the patient tissue via the jaw assembly 122. The controller 702 may be configured to apply the clamp pressure to the patient tissue using one of a number of different application routines. For example, in block 1006, the controller 702 may be configured to control the jaw assembly 122 to apply an increasing clamp pressure to the patient tissue. That is, the controller 702 may control the jaw assembly 122 to apply a clamp pressure that increases over time. In such embodiments, the clamp pressure may increase over time in any fashion (e.g., the clamp pressure may increase linearly, exponentially, gradually, or quickly over time or vary in its amount of increase over time). In other embodiments, in block 1008, the controller 702 may control the jaw assembly 122 to apply a random clamp pressure to the patient’s tissue. For example, the jaw assembly 122 may be controlled to apply a sequence of random or different clamp pressures to the patient’s tissue.

Regardless of the modality of clamp pressure used, the controller 702 is configured to determine the clamp pressure applied to the patient tissue in block 1010. To do so, the controller 702 may sample the sensor data produced by the clamp force sensor 804 at discrete times while the jaw assembly 122 applies the clamp pressure. For example, in embodiments in which an increasing clamp pressure is applied in block 1006, the controller 702 may sample the resulting clamp pressure, as indicated by the sensor data, at various times or continuously while the clamp pressure is being increased. Alternatively, in embodiments in which a sequence of random or different clamp pressures are applied in block 1008, the controller 702 may sample the resulting clamp pressure for each distinct clamp pressure of the sequence of clamp pressures.

In block 1012, the controller 702 also determines a corresponding tissue impedance for each clamp pressure determined in block 1010 using the RF electrode 802. To do so, in some embodiments, the controller 702 may be configured to apply a voltage to the patient tissue and determine a resulting current using the RF electrode 802. In this way, the controller 702 determines a tissue impedance correlated to each determined clamp pressure. By applying various or varying clamp pressures to the patient’s tissue, an array of tissue impedance value-to-clamp pressure value pairs can be generated. Accordingly, in block 1014, the controller 702 is configured to generate the tissue impedance fingerprint based on the determined clamp pressure values and the correlated tissue impedance values.

As discussed above, the tissue impedance fingerprint may be embodied as discrete tissue impedance-to-clamp pressure value pairs in some embodiments. Alternatively, the tissue impedance fingerprint may be embodied as, or otherwise stored as, a graph of tissue impedance-to-clamp pressure values. For example, a graph 1100 of a tissue impedance fingerprint 1102 is shown in FIG. 11. The tissue impedance fingerprint 1102 is formed from a set of measured tissue impedance-to-clamp pressure values 1110, which together form a graph (e.g., via interpolation of intervening values). It should be appreciated that the tissue impedance fingerprint 1102 may be unique to the patient’s tissue and different from other tissue impedance fingerprints 1104 determined from other patient’s tissue.

Referring back to FIG. 9, after the controller 702 has determined the tissue impedance fingerprint of the patient tissue grasped by the jaw assembly 122 using the method 1000, the method 900 advances to block 906. In block 906, the controller 702 may be configured to modify one or more operational characteristics of the energy-based surgical instrument 102 based on, or as a function of, the tissue impedance fingerprint determined in block 904. Any suitable operational characteristic of the energy-based surgical instrument 102 may be modified in block 906. For example, in some embodiments in block 908, the controller 702 may adjust a power level setting of the energy-based surgical instrument 102 based on the tissue impedance fingerprint. That is, the controller 702 may increase or decrease the amount of ultrasonic power provided by the harmonic blade 130 or the amount of RF energy applied by the RF electrode 802.

Additionally, in some embodiments in block 910, the controller may adjust energy application modalities of the energy-based surgical instrument 102 based on the tissue impedance fingerprint. For example, the controller 702 may determine to use harmonic energy rather than RF energy on the patient tissue or vice-versa. Alternatively, the controller 702 may determine the application length of each type of energy application. In some embodiments in block 912, the controller 702 may adjust one or more control points of the energy application of the energy-based surgical instrument 102 based on the determined tissue impedance fingerprint. The control points may dictate, for example, the level and length of various energy application routines. Additionally, in some embodiments in block 914, the controller 702 may adjust the clamp pressure timing of the jaw assembly 122 based on the determined tissue impedance fingerprint. The clamp pressure timing may be dictate the length of time the patient’s tissue is compressed by the jaw assembly 122 at various clamp pressures and/or the time at which such clamp pressure are applied to the patient tissue.

After the controller 702 has modified the operational characteristic of the energy-based surgical instrument 102 as a function of the tissue impedance fingerprint of the patient’s tissue in block 906, the surgeon may continue performing the surgical operation using the energy-based surgical instrument 102 in block 916. In block 918, the controller 702 determines whether an energy mode has been active. For example, the controller 702 may determine whether the surgeon has active an RF energy mode of the energy-based surgical instrument 102. If so, the method 900 advances to block 920 in which the controller 702 activates the energy mode based on the determined tissue impedance fingerprint of the patient’s tissue. That is, as discussed above, the controller 702 may be configured to adjust or modify the application of the energy mode as a function of the tissue impedance fingerprint. For example, in block 922, the controller 702 may adjust a power level setting of the energy mode based on the tissue impendence fingerprint. In this way, operation of the energy-based surgical instrument 102 is customized to the tissue of each patient by initially determining a tissue impedance fingerprint of the patient’s tissue and modifying the operational characteristics of the energy-based surgical instrument 102 based on the determine tissue impedance.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A method for operating an energy-based surgical instrument, the method comprising:
generating, by a controller of the energy-based surgical instrument, a tissue impedance fingerprint of patient tissue grasped within a jaw assembly of an end effector of the energy-based surgical instrument, wherein the tissue impedance fingerprint is indicative of an impedance response of the patient tissue to different amounts of clamp pressure on the patient tissue by the jaw assembly;
adjusting, by the controller, an operational characteristic of the energy-based surgical instrument as a function of the tissue impedance fingerprint; and
activating, by the controller, an operation of the energy-based surgical instrument using the adjusted operational characteristic.

2. The method of claim 1, wherein generating the tissue impedance fingerprint comprises:
determining, by the controller, a clamp pressure exerted on the patient tissue by the jaw assembly; and
determining, by the controller, an impedance value of the patient tissue at the clamp pressure exerted on the patient tissue by the jaw assembly.

3. The method of claim 2, wherein determining the clamp pressure comprises:
applying, by the jaw assembly, an increasing clamp pressure on the patient tissue over a time period; and
determining, by the controller, a clamp pressure exerted on the patient tissue by the jaw assembly at multiple points in time within the time period.

4. The method of claim 3, wherein determining the impedance value of the patient tissue comprises determining, by the controller, an impedance value of the patient tissue at each point in time correlating to a determined clamp pressure.

5. The method of claim 2, wherein determining the clamp pressure comprises:
applying, by the jaw assembly, a sequence of random clamp pressures on the patient tissue of the patient tissue; and
determining, by the controller, a clamp pressure exerted on the patient tissue by the jaw assembly for each random clamp pressure of the sequence of random clamp pressures.

6. The method of claim 5, wherein determining the impedance value of the patient tissue comprises determining, by the controller, an impedance value of the patient tissue for each random clamp pressure of the sequence of random clamp pressures.

7. The method of claim 2, wherein determining the clamp pressure comprises:
receiving, by the controller, sensor data from a clamp force sensor of the energy-based surgical instrument, the sensor data being indicative of the clamp pressure; and
determining, by the controller, the clamp pressure exerted on the patient tissue by the jaw assembly based on the sensor data.

8. The method of claim 1, wherein adjusting the operational characteristic of the energy-based surgical instrument comprises adjusting a power level setting of the energy-based surgical instrument as a function of the tissue impedance fingerprint.

9. The method of claim 1, wherein adjusting the operational characteristic of the energy-based surgical instrument comprises adjusting an energy application modality of the energy-based surgical instrument as a function of the tissue impedance fingerprint.

10. The method of claim 1, wherein adjusting the operational characteristic of the energy-based surgical instrument comprises adjusting a control point of an application of energy from the energy-based surgical instrument as a function of the tissue impedance fingerprint.

11. The method of claim 1, wherein adjusting the operational characteristic of the energy-based surgical instrument comprises adjusting a length of time for which the jaw assembly applies a clamp pressure to the patient tissue as a function of the tissue impedance fingerprint.

12. The method of claim 1, wherein adjusting the operational characteristic of the energy-based surgical instrument comprises adjusting a power level setting of an energy application of the energy-based surgical instrument as a function of the tissue impedance fingerprint while the energy application is being performed.

13. An energy-based surgical instrument comprising:
an end effector having a jaw assembly configured to grasp patient tissue;
a clamp force sensor configured to generate sensor data indicative of an amount of clamp pressure applied by the jaw assembly on the patient tissue when the patient tissue is grasped by the jaw assembly; and
a controller configured to:
generate a tissue impedance fingerprint of the patient tissue, wherein the tissue impedance fingerprint is indicative of an impedance response of the patient tissue to different amounts of clamp pressure on the patient tissue by the jaw assembly;
adjust an operational characteristic of the energy-based surgical instrument as a function of the tissue impedance fingerprint; and
activate an operation of the energy-based surgical instrument using the adjusted operational characteristic.

14. The energy-based surgical instrument of claim 13, wherein to generate the tissue impedance fingerprint comprises to:
determine a clamp pressure exerted on the patient tissue by the jaw assembly based on the sensor data; and
determine an impedance value of the patient tissue at the clamp pressure exerted on the patient tissue by the jaw assembly.

15. The energy-based surgical instrument of claim 14, wherein to determine the clamp pressure comprises to:
control the jaw assembly to apply, by the jaw assembly, an increasing clamp pressure on the patient tissue over a time period; and
determine a clamp pressure exerted on the patient tissue by the jaw assembly at multiple points in time within the time period based on the sensor data.

16. The energy-based surgical instrument of claim 15, wherein to determine the impedance value of the patient tissue comprises to determine an impedance value of the patient tissue at each point in time correlating to a determined clamp pressure.

17. The energy-based surgical instrument of claim 14, wherein to determine the clamp pressure comprises to:
control the jaw assembly to apply, by the jaw assembly, a sequence of random clamp pressures on the patient tissue of the patient tissue; and
determine a clamp pressure exerted on the patient tissue by the jaw assembly for each random clamp pressure of the sequence of random clamp pressures based on the sensor data.

18. The energy-based surgical instrument of claim 17, wherein to determine the impedance value of the patient tissue comprises to determine an impedance value of the patient tissue for each random clamp pressure of the sequence of random clamp pressures.

19. The energy-based surgical instrument of claim 13, wherein to adjust the operational characteristic of the energy-based surgical instrument comprises to adjust a power level setting of the energy-based surgical instrument as a function of the tissue impedance fingerprint.

20. The energy-based surgical instrument of claim 13, wherein to adjust the operational characteristic of the energy-based surgical instrument comprises to adjust an energy application modality of the energy-based surgical instrument as a function of the tissue impedance fingerprint.

21. The energy-based surgical instrument of claim 13, wherein to adjust the operational characteristic of the energy-based surgical instrument comprises to adjust a control point of an application of energy from the energy-based surgical instrument as a function of the tissue impedance fingerprint.

22. The energy-based surgical instrument of claim 13, wherein to adjust the operational characteristic of the energy-based surgical instrument comprises to adjust a length of time for which the jaw assembly applies a clamp pressure to the patient tissue as a function of the tissue impedance fingerprint.

23. The energy-based surgical instrument of claim 13, wherein to adjust the operational characteristic of the energy-based surgical instrument comprises to adjust a power level setting of an energy application of the energy-based surgical instrument as a function of the tissue impedance fingerprint while the energy application is being performed.
